(19) United States
(12) Patent Application Publication
(10) Pub. No.: US 2022/0056221 A1
(43) Pub. Date: Feb. 24, 2022

(54) Method for Manufacturing Fibroin Solution and Method for Manufacturing Protein Molded Body

(71) Applicant: Spiber Inc., Yamagata (JP)

(72) Inventors: Hisahiro Kudo, Tsuruoka-shi, Yamagata (JP); Hideto Ishii, Tsuruoka-shi, Yamagata (JP); Tatsuki Adachi, Tsuruoka-shi, Yamagata (JP); Ryoji Okada, Tsuruoka-shi, Yamagata (JP)

(73) Assignee: Spiber Inc., Yamagata (JP)

(21) Appl. No.: 17/274,557

(22) PCT Filed: Sep. 13, 2019
(86) PCT No.: PCT/JP2019/036241

§ 371 Date: Mar. 9, 2021

(30) Foreign Application Priority Data
Sep. 14, 2018 (JP) 2018-172728

(51) Int. Cl. C08J 3/09; C07K 14/435; D01D 1/02; D01F 4/02

(52) U.S. Cl. CPC C08J 3/09; C07K 14/43518; C08J 2389/00; D01F 4/02; D01D 1/02

(57) ABSTRACT

Disclosed is a method for manufacturing a fibroin solution including a step of forming a slurry containing a solvent-containing dissolving liquid and a fibroin-containing protein powder dispersed in the dissolving liquid by continuously introducing the protein powder to a thin film of the dissolving liquid while flowing the thin film; and a step of forming a fibroin solution by dissolving, in the dissolving liquid, the protein powder in the slurry.

### TECHNICAL FIELD

The present invention relates to a method for manufacturing a fibroin solution and a method for manufacturing a protein molded body.

### BACKGROUND ART

Genetically modified fibroin is expected to be used as a material that can replace petroleum resources. For example, Patent Literature 1 discloses a technique for producing a fiber using, as a spinning raw liquid, a fibroin solution obtained by dissolving genetically modified fibroin in an appropriate solvent.

With regard to methods for manufacturing solutions of polymer materials, for example, Patent Literature 2 teaches a method involving dissolving an acrylonitrile-based polymer after producing a slurry in which the acrylonitrile-based polymer is dispersed. Patent Literature 2 also mentions that the slurry is obtained with generation of lumps suppressed by reducing the solubility of the acrylonitrile-based polymer by cooling the solvent. Patent Literature 3 discloses that the dissolving capacity of a solvent is reduced in a method for preparing a spinning solution for manufacturing acrylic fibers.

### CITATION LIST

### Patent Literature

### Patent Literature 1: JP 2014-129639 A

### Patent Literature 2: JP 2015-078451 A

### Patent Literature 3: JP 2015-132040 A

### SUMMARY OF INVENTION

### Technical Problem

It is expected that a high-concentration fibroin solution can be efficiently manufactured by a method involving dispersing a fibroin-containing powder in a solvent to obtain a slurry and then dissolving the powder in the solvent. However, this method may have a problem of generation of lumps in the step of forming the slurry.

However, a practical method for sufficiently suppressing generation of lumps in the step of forming a slurry in the manufacture of a fibroin solution is unknown so far. For fibroin, practical solvents are limited to high-melting-point solvents, such as dimethyl sulfoxide (DMSO) and formic acid, which makes it difficult to use the method in which the solubility is controlled by cooling the solvent as mentioned in Patent Literature 2. In addition, the use of the solvent together with a poor solvent for reducing the solubility in the solvent tends to easily generate a gel. There is also problem in which the use of a poor solvent requires heating at high temperature for dissolving fibroin in a solvent containing the poor solvent after forming a slurry. Heating at high temperature may degrade the physical properties of a protein molded body.

An object of one aspect of the present invention is to provide a method for effectively manufacturing a fibroin solution that may provide a protein molded body with good physical properties while suppressing generation of lumps in forming a slurry.

### Solution to Problem

In one aspect of the present invention, a method for manufacturing a fibroin solution includes:

a step of forming a slurry containing a solvent-containing dissolving liquid and a fibroin-containing protein powder dispersed in the dissolving liquid by continuously introducing the protein powder to a thin film of the dissolving liquid while flowing the thin film; and

a step of producing a fibroin solution by dissolving, in the dissolving liquid, the protein powder in the slurry.

In another aspect of the present invention, a method for manufacturing a protein molded body includes producing a fibroin solution by the above method and producing a fibroin-containing protein molded body by molding using the fibroin solution as a raw liquid for molding.

### Advantageous Effects of Invention

In a method according to one aspect of the present invention, a fibroin solution that may provide a protein molded body with good physical properties can be continuously and effectively manufactured with generation of lumps suppressed in forming a slurry.

### BRIEF DESCRIPTION OF DRAWINGS

FIG. 1 is a schematic view of one embodiment of a step of forming a slurry containing a protein powder.

FIG. 2 is a schematic view of one embodiment of the step of forming a slurry containing a protein powder.

FIG. 3 is a schematic view of one embodiment of the step of forming a slurry containing a protein powder.

FIG. 4 is a schematic view of one embodiment of a step of forming a fibroin solution from the slurry.

### DESCRIPTION OF EMBODIMENTS

Some embodiments of the present invention will be described below in detail. However, the present invention is not limited to the following embodiments.

(Method for Manufacturing Fibroin Solution)

One embodiment of a method for manufacturing a fibroin solution includes a dispersion step of forming a slurry by continuously introducing a fibroin-containing protein powder to a thin film of a dissolving liquid while flowing the thin film; and a dissolution step of producing a fibroin solution by dissolving, in the dissolving liquid, the protein powder in the slurry.

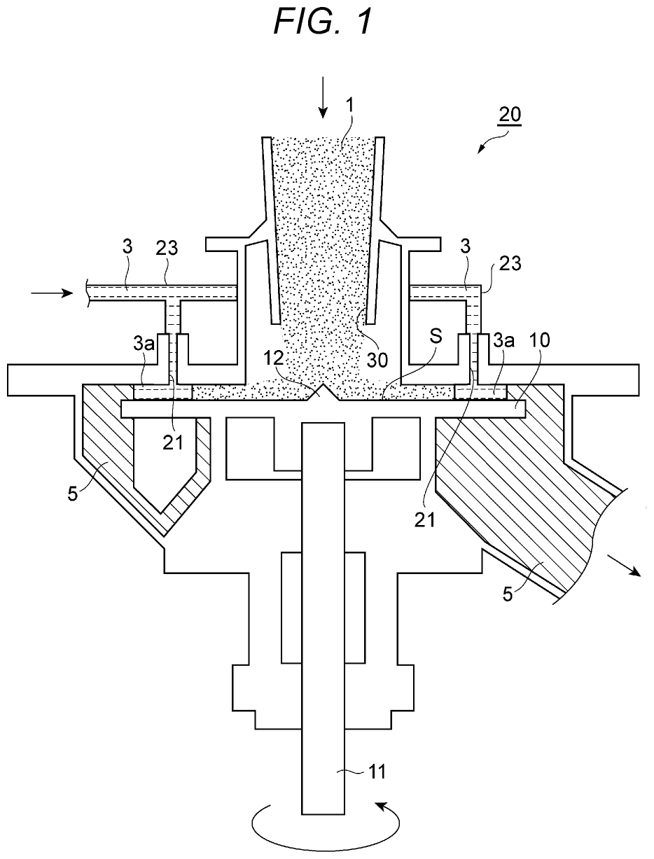

FIG. 1 is a schematic view of one embodiment of a step (dispersion step) of forming a slurry containing a protein powder. In the method shown in FIG. 1, a mixing device 20 including a plate-shaped rotary body 10 is used. The rotary body 10 has a rotation surface S and can be driven to rotate. With the rotary body 10 rotating, a protein powder 1 and a dissolving liquid 3 are each supplied onto the rotation surface S of the rotary body 10. The dissolving liquid 3 forms a flowing thin film 3a when the dissolving liquid 3 flows down in the radial direction on the rotation surface S. The flowing thin film 3a and the protein powder 1 are mixed on the rotation surface S. A slurry 5 formed as a result contains the dissolving liquid and the protein powder dispersed in the dissolving liquid. In FIG. 1, a clear boundary between the thin film 3a of the dissolving liquid and the slurry 5 is depicted for easy understanding. However, in general, a clear boundary is not formed between the thin film 3a and the slurry 5 because the thin film 3a to which the protein powder has been introduced gradually forms the slurry 5 while the thin film 3a flows.

The rotary body 10 has a disc shape. The rotation surface S is a circular plane. The rotation surface S has a conical controller 12 at its central portion. The controller 12 controls the flow of the powder. The shape of the rotary body is not limited to this. For example, the rotation surface may have a conical shape. In this case, the thin film of the dissolving liquid normally flows in a spiral manner. The rotary body may have a cylindrical shape. The rotation surface may have unevenness as desired. Like the embodiment in FIG. 1, the circular rotation surface S and the disc-shaped rotary body 10 having the controller 12 are advantageous in dispersion efficiency, processing efficiency, and device energy consumption. The rotary body 10 is driven to rotate by a shaft 11 connected to a driving device, such as a motor. The rotational speed of the rotary body 10 is not particularly limited, but may be, for example, 50 to 1000 rpm. The width of the rotation surface S is not particularly limited, but may be, for example, 50 to 1000 mm.

In the embodiment in FIG. 1, the dissolving liquid 3 moves in a feed pipe 23 connected to two dissolving liquid inlets 21 disposed adjacent to the rotation surface S. The dissolving liquid 3 is next supplied to the rotation surface S through the dissolving liquid inlets 21. The supplied dissolving liquid 3 forms the thin film 3a flowing on the rotation surface S. The formed thin film 3a of the dissolving liquid 3 immediately meets the protein powder 1 flowing on the rotation surface S from its central portion in the radial direction. The protein powder 1 is accordingly introduced to the flowing thin film 3a. The introduction of the protein powder 1 to the flowing thin film 3a of the dissolving liquid 3 allows the protein powder 1 to be readily uniformly dispersed without concentrating in a particular area. As a result, the protein powder 1 can be effectively dispersed in the dissolving liquid 3 with generation of lumps suppressed.

The dissolving liquid 3 contains a solvent for dissolving fibroin. With the dissolving liquid 3 being fed with a general-purpose pump, the flow rate of the dissolving liquid 3 may be measured and controlled. To prevent fluctuations in flow rate, the dissolving liquid 3 may be continuously supplied with a metering pump. Examples of the metering pump include gear pumps and uniaxial eccentric screw pumps. Gear pumps are superior in flow rate control accuracy. The flow rate of the dissolving liquid 3 may be, for example, 1 to 3000 kg/hr.

The solvent in the dissolving liquid 3 may contain, for example, at least one selected from the group consisting of hexafluoroisopropanol (HFIP), hexafluoroacetone (HFA), dimethyl sulfoxide (DMSO), N,N-dimethylformamide (DMF), and formic acid. From the viewpoint of fibroin solubility, the solvent may be dimethyl sulfoxide, formic acid, hexafluoroisopropanol (HFIP), or a mixed solvent containing a combination thereof.

The dissolving liquid 3 may further contain one or two or more inorganic salts dissolved in the solvent. Inorganic salts can promote the dissolution of fibroin. Examples of inorganic salts include inorganic salts composed of the following Lewis acid and Lewis base. Examples of the Lewis base include an oxo acid ion (nitrate ion, perchlorate ion, or the like), a metal oxo acid ion (permanganate ion or the like), a halide ion, a thiocyanate ion, and a cyanate ion. Examples of the Lewis acid include a metal ion such as an alkali metal ion or an alkaline earth metal ion, a polyatomic ion such as an ammonium ion, and a complex ion.

Examples of inorganic salts include lithium salts such as lithium chloride, lithium bromide, lithium iodide, lithium nitrate, lithium perchlorate, and lithium thiocyanate; calcium salts such as calcium chloride, calcium bromide, calcium iodide, calcium nitrate, calcium perchlorate, and calcium thiocyanate; iron salts such as iron chloride, iron bromide, iron iodide, iron nitrate, iron perchlorate, and iron thiocyanate; aluminum salts such as aluminum chloride, aluminum bromide, aluminum iodide, aluminum nitrate, aluminum perchlorate, and aluminum thiocyanate; potassium salts such as potassium chloride, potassium bromide, potassium iodide, potassium nitrate, potassium perchlorate, and potassium thiocyanate; sodium salts such as sodium chloride, sodium bromide, sodium iodide, sodium nitrate, sodium perchlorate, and sodium thiocyanate; zinc salts such as zinc chloride, zinc bromide, zinc iodide, zinc nitrate, zinc perchlorate, and zinc thiocyanate; magnesium salts such as magnesium chloride, magnesium bromide, magnesium iodide, magnesium nitrate, magnesium perchlorate, and magnesium thiocyanate; barium salts such as barium chloride, barium bromide, barium iodide, barium nitrate, barium perchlorate, and barium thiocyanate; and strontium salts such as strontium chloride, strontium bromide, strontium iodide, strontium nitrate, strontium perchlorate, and strontium thiocyanate. The inorganic salt may be lithium chloride, calcium chloride, or a combination thereof.

The content of the inorganic salt with respect to the total mass of the dissolving liquid may be 0.1% by mass or more, 1% by mass or more, 4% by mass or more, 7% by mass or more, 10% by mass or more, or 15% by mass or more, and may be 20% by mass or less, 16% by mass or less, 12% by mass or less, or 9% by mass or less. The content of the inorganic salt with respect to the total mass of the dissolving liquid may be 0.1% by mass or more and 20% by mass or less, 16% by mass or less, 12% by mass or less, or 9% by mass or less, may be 1% by mass or more and 20% by mass or less, 16% by mass or less, 12% by mass or less, or 9% by mass or less, may be 4% by mass or more and 20% by mass or less, 16% by mass or less, 12% by mass or less, or 9% by mass or less, may be 7% by mass or more and 20% by mass or less, 16% by mass or less, 12% by mass or less, or 9% by mass or less, may be 10% by mass or more and 20% by mass or less, 16% by mass or less, 12% by mass or less, or 9% by mass or less, or may be 15% by mass or more and 20% by mass or less, 16% by mass or less, 12% by mass or less, or 9% by mass or less.

By using, for example, a feeder, the protein powder 1 is continuously supplied toward a central portion of the rotation surface S through a powder inlet 30 disposed above the rotation surface S. The protein powder 1 is accordingly introduced to the thin film of the dissolving liquid 3 without coming into contact with an area wetted with the dissolving liquid. When the protein powder before being introduced to the thin film of the dissolving liquid comes into contact with a small amount of dissolving liquid on the rotary body, the protein powder adheres to the rotary body or the like, which may hinder continuous dispersion.

The feeder for supplying the protein powder 1 may be a metering feeder. The use of a metering feeder makes it easy to produce a fibroin solution having a constant concentration of fibroin. Fluctuations in fibroin concentration may significantly affect the quality of protein molded bodies such as protein fibers. The supply rate of the protein powder 1 may be, for example, 1 to 1000 kg/hr.

The shape of individual primary particles constituting the protein powder 1 is not particularly limited. The primary particles may be in the form of particles. If the primary particles have a small particle size, lumps tend to be generated. Even in such a case, generation of lumps can be suppressed according to this embodiment.

During the process for forming the slurry 5, heat may be generated. For this, the mixing device may be cooled, or the dissolving liquid 3 to be supplied may be cooled in advance. The mixing device can be cooled with, for example, a jacket surrounding the device. The cooling temperature of the jacket and the solvent may be −20° C. or more and 30° C. or less, −10° C. or more and 20° C. or less, 0° C. or more and 15° C. or less, or 5° C. or more and 10° C. or less.

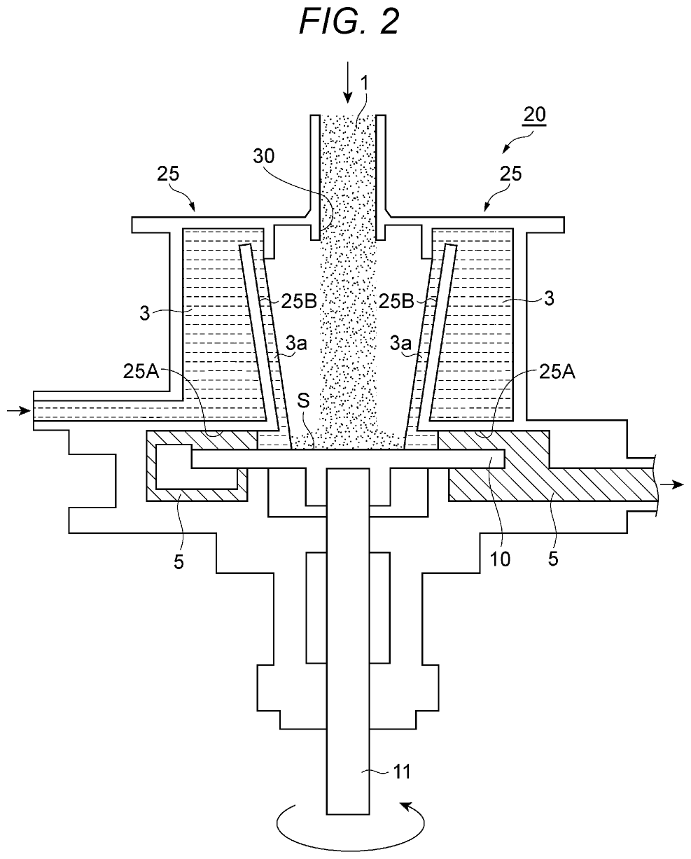

FIG. 2 is a schematic view of another embodiment of the step (dispersion step) of forming a slurry containing a protein powder. A mixing device 20 shown in FIG. 2 has an annular retention tank 25 disposed above a rotation surface S. The retention tank 25 has a bottom surface 25A and an inner circumferential surface 25B. The bottom surface 25A faces the rotation surface S. The inner circumferential surface 25B forms an opening above a central portion of the rotation surface S. A dissolving liquid 3 is retained in the retention tank 25, then flows down on the inner circumferential surface 25B from above the inner circumferential surface 25B while forming a thin film 3a, and is supplied onto the rotation surface S below the inner circumferential surface 25B. A protein powder 1 is continuously supplied toward a central portion of the rotation surface S through a powder inlet 30 disposed above the rotation surface S and meets the thin film of the dissolving liquid 3 on the rotation surface S at a position below the inner circumferential surface 25B.

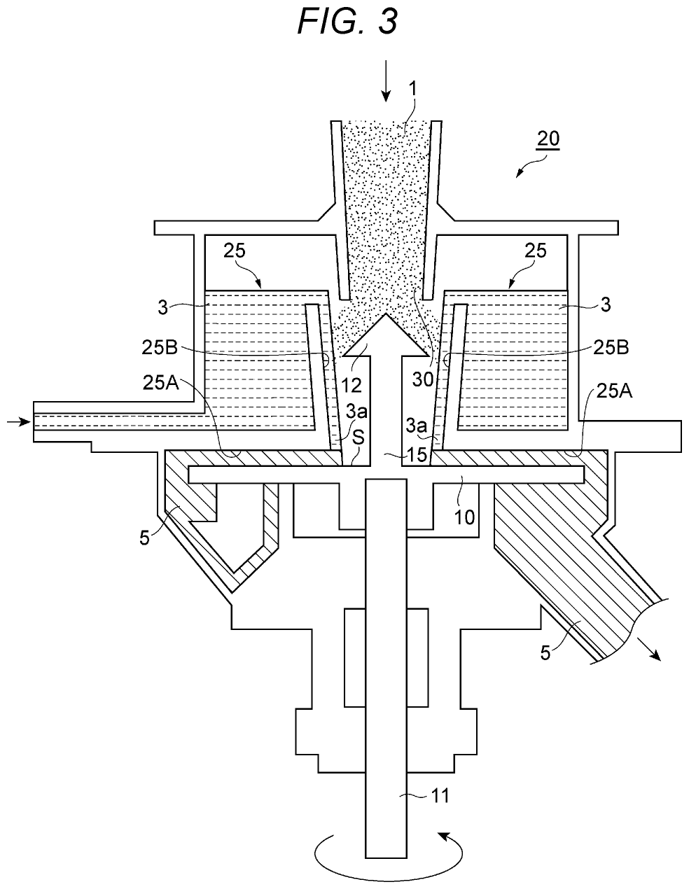

FIG. 3 is also a schematic view of another embodiment of the step (dispersion step) of forming a slurry containing a protein powder. A mixing device 20 shown in FIG. 3 has an annular retention tank 25 disposed above a rotation surface S. The retention tank 25 has a bottom surface 25A and an inner circumferential surface 25B. The bottom surface 25A faces the rotation surface S. The inner circumferential surface 25B forms an opening above a central portion of the rotation surface S. A dissolving liquid 3 is retained in the retention tank 25, then flows down on the inner circumferential surface 25B from above the inner circumferential surface 25B while forming a thin film 3a, and is supplied onto the rotation surface S below the inner circumferential surface 25B.

The mixing device 20 in FIG. 3 further has a columnar portion 15. The columnar portion 15 extends from a central portion of the rotation surface S toward a powder inlet 30. The columnar portion 15 has a conical controller 12 at its end. The controller 12 controls the flow of the protein powder 1. A protein powder 1 is continuously supplied toward the controller 12 through the powder inlet 30. The protein powder 1 flowing in directions changed by the controller 12 meets the thin film 3a flowing on the inner circumferential surface 25B before the protein powder 1 reaches the rotation surface S. The protein powder 1 is accordingly introduced to the thin film 3a on the rotation surface S of the dissolving liquid 3.

The other structures of the embodiments in FIG. 2 and FIG. 3 are the same as those of the embodiment in FIG. 1.

Examples of the mixing device used in the method illustrated above include a flow jet mixer (model MW-J-300) available from Funken Powtechs, Inc.

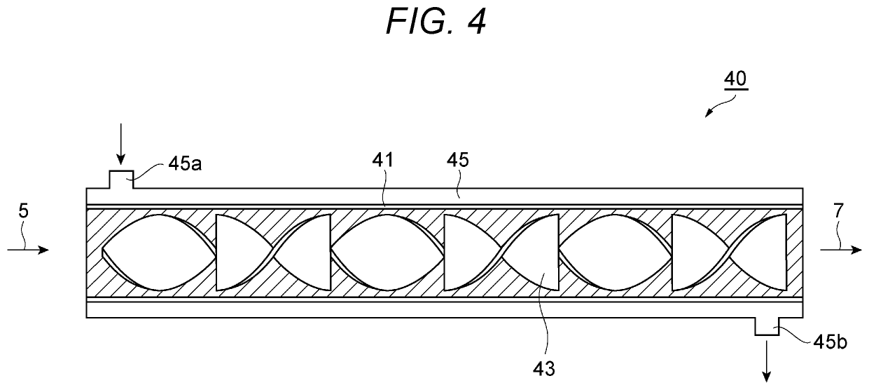

FIG. 4 is a schematic view of one embodiment of a step (dissolution step) of forming a fibroin solution from a slurry. An in-line mixer 40 shown in FIG. 4 has a pipe 41, a static mixer element 43 accommodated inside the pipe 41, and a jacket 45 covering the outer circumferential surface of the pipe 41. The jacket 45 has a heating medium inlet 45a and a heating medium outlet 45b. A heating medium that has been heated to a predetermined temperature flows from the heating medium inlet 45a to the heating medium outlet 45b. A slurry 5 passing through the in-line mixer 40 is heated accordingly.

The slurry 5 is introduced from one end of the pipe 41. The slurry 5 is stirred with the static mixer element 43 in the pipe 41 and heated with the heat transferred from the jacket 45. The protein powder in the slurry 5 is accordingly dissolved in the dissolving liquid to form a fibroin solution 7. The formed fibroin solution 7 is discharged from the other end of the pipe 41.

The temperature (heating temperature) of the heating medium introduced into the jacket 45 is adjusted such that the fibroin solution discharged from the in-line mixer reaches an intended temperature. The temperature of the fibroin solution 7 discharged from the in-line mixer 40 may be 20° C. or more and 120° C. or less, 30° C. or more and 100° C. or less, 40° C. or more and 90° C. or less, or 50° C. or more and 80° C. or less. If this temperature is below 20° C., more fibroin tends to remain undissolved. If this temperature is above 120° C., fibroin may thermally decompose.

The inner diameter and length of the pipe 41 of the in-line mixer 40 and the number of static mixer elements 43 are selected such that the protein powder dissolves well in the dissolving liquid. The time it takes for the slurry 5 to pass through the pipe 41, or the dissolution time, is 10 seconds or more and 300 seconds or less, 20 seconds or more and 200 seconds or less, 30 seconds or more and 120 seconds or less, or 40 seconds or more and 90 seconds or less. When the dissolution time is 10 seconds or more, it is easy to sufficiently dissolve the protein powder. A dissolution time of 300 seconds is normally adequate for sufficient dissolution. An exceedingly long dissolution time requires an unnecessarily huge device.

The method for dissolving, in the dissolving liquid, the protein powder in the slurry is not limited to the method using the in-line mixer illustrated in FIG. 4. For example, the protein powder may be dissolved in the dissolving liquid by heating in a stirring vessel, addition of a solvent, use of a mixed solvent, addition of a salt, or a combination thereof.

The method for manufacturing a fibroin solution may further include a step of removing insoluble matter by filtering the fibroin solution. The fibroin solution discharged from the in-line mixer in the dissolution step may be filtered through a filter disposed downstream of the in-line mixer. When the fibroin solution is filtered while it has high temperature, the pressure drop is small because of low viscosity of the solution.

The method for manufacturing a fibroin solution may further include a step of defoaming the fibroin solution. The fibroin solution discharged from the in-line mixer in the dissolution step may be defoamed by a defoaming device disposed downstream of the in-line mixer. When the fibroin solution is filtered while it has high temperature, the speed of moving bubbles is high because of low viscosity of the solution. Examples of the defoaming method include a method involving enclosing the fibroin solution inside a sealed container and depressurizing the sealed container, a method involving forming the fibroin solution into a thin film under reduced pressure, and a method involving centrifugal gas-liquid separation.

The method for manufacturing a fibroin solution may further include a step of cooling the fibroin solution under stirring in the in-line mixer. When the method for manufacturing a fibroin solution includes a step of removing insoluble matter from the fibroin solution and/or a step of defoaming the fibroin solution, the fibroin solution may be cooled after these steps. The temperature of the fibroin solution after cooling may be 0° C. or more and 40° C. or less. Although the viscosity of the fibroin solution greatly varies depending on temperature, the fibroin solution tends to be easily fed as long as the fibroin solution has a temperature of 10° C. or more. As long as the fibroin solution has a temperature of 40° C. or less, it is easy to sufficiently suppress decomposition of fibroin even when the fibroin solution is retained for a long period of time. Suppressing decomposition of fibroin provides a protein molded body having better physical properties. From the same viewpoint, the temperature of the fibroin solution after cooling may be 5° C. or more and 30° C. or less, 10° C. or more and 25° C. or less, or 15° C. or more and 20° C. or less.

The concentration of fibroin in the manufactured fibroin solution is not particularly limited, but may be, for example, 5% to 50% by mass, 10% to 50% by mass, 20% to 50% by mass, or 25% to 50% by mass based on the mass of the fibroin solution. The fibroin solution having a high concentration of fibroin can have better spinnability and, when being formed into a fiber being a protein molded body, enables drawing at a higher draw ratio. With a higher draw ratio, it is possible to obtain a fiber having high strength. Since lump generation and the amount of undissolved matter are small in the method according to this embodiment, it is easy to obtain a high-concentration fibroin solution. The manufactured fibroin solution may be directly subjected to the molding step or may be stored in a storage tank.

(Method for Manufacturing Protein Molded Body)

A protein molded body containing fibroin can be manufactured by molding using, as a raw liquid for molding, the fibroin solution obtained by the method according to the embodiment. The manufactured protein molded body may be, for example, a film or fiber.

The manufactured protein molded body is a molded body containing fibroin as a main component. The proportion of fibroin in the total mass of the protein molded body may be 50% by mass or more, 60% by mass or more, 65% by mass or more, 70% by mass or more, 75% by mass or more, 80% by mass or more, or 90% by mass or more, or may be 100% by mass or less.

A protein fiber can be manufactured by spinning using the fibroin solution as a spinning raw liquid. The spinning step, which is an example of the molding step, will be described below.

The fibroin solution serving as a spinning raw liquid may further contain various additives as necessary. Examples of additives include plasticizers, leveling agents, cross-linking agents, crystal nucleating agents, antioxidants, ultraviolet absorbers, coloring agents, fillers, and synthetic resins. The content of the additives may be 0 parts by mass or more and 50 parts by mass or less with respect to 100 parts by mass of the total amount of fibroin in the spinning raw liquid.

In the spinning step, the fibroin solution is extruded from a spinneret and next formed into a fiber shape by removing the solvent from the fibroin solution. The fibroin solution may be fed to the spinneret from the storage tank by using a metering pump. The shape of the spinneret, the hole shape, the number of holes, and the like are not particularly limited and can be appropriately selected according to a desired fiber diameter and the number of filaments.

When the spinneret has a circular hole shape, the hole diameter may be 0.03 mm or more and 0.6 mm or less. With a hole diameter of 0.03 mm or more, it is possible to reduce the pressure drop and the equipment cost. With a hole diameter of 0.6 mm or less, the need of the drawing operation for reducing the fiber diameter is reduced, which makes it possible to reduce the possibility of drawing breakage between extrusion and take-up.

The temperature of the fibroin solution passing through the spinneret and the temperature of the spinneret are not particularly limited, and appropriately adjusted by, for example, the concentration and viscosity of fibroin in the fibroin solution and the type of solvent. The temperature of the fibroin solution passing through the spinneret may be 0° C. to 70° C. in order to prevent, for example, degradation of fibroin. The temperature of the fibroin solution passing through the spinneret may be below the boiling point of the solvent in order to reduce an increase in pressure caused by volatilization of the solvent and the possibility of generation of blockage in the pipe caused by solidification of the spinning raw liquid. This improves the process stability.

The method for removing the solvent of the fibroin solution extruded from the spinneret is not particularly limited. For example, the solvent may be volatilized by hot air in a gas phase. The solvent may be removed in a liquid phase containing a poor solvent that may be mixed with the solvent of the fibroin solution without dissolving fibroin. When the solvent of the fibroin solution is removed in a liquid phase, the fibroin solution may be extruded from the spinneret into the liquid phase through the gas phase or may be directly extruded into the liquid phase without passing through the gas phase.

A fiber formed by removal of the solvent may be drawn. The draw ratio based on the time point at which the formed fiber passes through the first take-up roller may be 2 times or more and 30 times or less, 15 times or less, or 10 times or less, may be 3 times or more and 30 times or less, 15 times or less, or 10 times or less, may be 4 times or more and 30 times or less, 15 times or less, or 10 times or less, may be 4 times or more and 30 times or less, 15 times or less, or 10 times or less, may be 5 times or more and 30 times or less, 15 times or less, or 10 times or less, or may be 6 times or more and 30 times or less, 15 times or less, or 10 times or less. The method according to this embodiment tends to enable the fibroin solution formed with generation of lumps suppressed to undergo spinning at higher draw ratio. The draw ratio is adjusted within a range in which desired fiber thickness and characteristics such as mechanical properties are obtained. Drawing may be performed once or may be performed stepwise several times. Drawing may be performed in liquid or may be performed after drying.

As necessary, an oil for providing bundle compactness, charge suppression, and lubricity may be applied to the formed protein fiber as desired before drying and winding. The type, amount, and the like of applied oil are not particularly limited and can be appropriately adjusted in consideration of, for example, application and handleability of fiber.

The formed protein fiber is dried by a usual method. Subsequently, the protein fiber may be wound by a winder. The winding conditions, such as suitable tension and contact pressure, in the winder are freely adjusted.

A fibroin film can be obtained by, for example, a method including subjecting the fibroin solution, which is used as a dope solution, to cast molding on the surface of a base member and drying the formed coating film and/or performing solvent removal.

The viscosity of the dope solution for manufacturing the fibroin film may be 15 to 80 cP (centipoise) or 20 to 70 cP. The concentration of fibroin may be 3% to 50% by mass, 3.5% to 35% by mass, or 4.2% to 15.8% by mass with respect to 100% by mass of the total amount of the dope solution.

The base member may be, for example, a resin substrate, a glass substrate, or a metal substrate. The base member may be a resin substrate since it is easy to peel a formed film. The resin substrate may be, for example, a polyethylene terephthalate (PET) film, a fluororesin film such as a polytetrafluoroethylene film, a polypropylene (PP) film, or a release film having a silicone compound immobilized on the surface of such a film. The base member may be a release film having a silicone compound immobilized in a PET film or on the surface of a PET film because the release film is stable against solvents such as HFIP and DMSO, the dope solution can stably undergo cast molding, and it is easy to peel a film after molding.

An example of cast molding includes casting a dope liquid onto the surface of a base member and adjusting the thickness of the coating film to a predetermined thickness by using a film thickness control means, such as an applicator, a knife coater, and a bar coater. The predetermined thickness is set such that the thickness of the coating film after drying and/or solvent removal is, for example, 1 to 1000 μm.

The drying and solvent removal of the coating film can be carried out by a dry method or a wet method. Examples of the dry method include vacuum drying, hot-air drying, and air drying. Examples of the wet method include immersion of the coating film in a solvent removal liquid. Examples of the solvent removal liquid include water, C1-C5 lower alcohols, such as methanol, ethanol, and 2-propanol, and a mixture of water and an alcohol. The temperature of the solvent removal liquid may be 0° C. to 90° C.

After drying and/or solvent removal, the formed fibroin film may be uniaxially drawn or biaxially drawn in water. Biaxial drawing may be sequential drawing or simultaneous biaxial drawing. Multi-stage drawing of two or more stages may be performed. The draw ratio may be 1.01 to 6 times, or 1.05 to 4 times both in length and width. With the draw ratio within this range, it is easy to balance stress and strain. The drawing in water can be performed at a water temperature of, for example, 20° C. to 90° C. The fibroin film after drawing may undergo heat setting under dry heating at 50° C. to 200° C. for 5 to 600 seconds. This heat setting provides a fibroin film having good dimensional stability at room temperature. In general, a uniaxially drawn film is a uniaxially oriented film, and a biaxially drawn film is a biaxially oriented film.

(Fibroin)

Fibroin used for manufacturing a fibroin solution and a protein molded body may be natural fibroin or may be modified fibroin derived from natural fibroin. Fibroin may be fibroin manufactured from microorganisms or the like by genetic recombination technology, may be chemically synthesized fibroin, or may be purified naturally-occurring fibroin.

Fibroin may be, for example, one or more selected from the group consisting of silk fibroin, spider silk fibroin, and hornet silk fibroin. In particular, fibroin may be silk fibroin, spider silk fibroin, or a combination thereof. In combination of silk fibroin and spider silk fibroin, the proportion of silk fibroin is, for example, 40 parts by mass or less, 30 parts by mass or less, or 10 parts by mass or less with respect to 100 parts by mass of spider silk fibroin.

Silk fibroin may be sericin-removed silk fibroin, sericin-unremoved silk fibroin, or a combination thereof. Sericin-removed silk fibroin is produced by purifying silk fibroin by removing sericin covering silk fibroin, other fats, and the like. The purified silk fibroin may be a freeze-dried powder. Sericin-unremoved silk fibroin is unpurified silk fibroin from which sericin and the like have not been removed.

Spider silk fibroin may be natural spider silk fibroin, or modified spider silk fibroin (artificial spider silk fibroin) derived from natural spider silk fibroin.

Examples of natural spider silk fibroin include large spinneret dragline silk proteins, weft thread silk proteins, and minor ampullate gland silk proteins. Large spinneret dragline silk proteins have both high stress and stretchability because they have a repeated region consisting of a crystalline region and a noncrystalline region (also referred to as an amorphous region). Weft thread silk proteins have a characteristic of not having a crystalline region but having a repeated region consisting of a non-crystalline region. Weft thread silk proteins are inferior in stress as compared with large spinneret dragline silk proteins, but have high stretchability.

Large spinneret dragline silk proteins are also characterized by having excellent toughness because they are produced from the major ampullate gland. Examples of large spinneret dragline silk proteins include major ampullate spidroins MaSp1 and MaSp2 derived from Nephila clavipes, and ADF3 and ADF4 derived from Araneus diadematus. ADF3 is one of the two major dragline silk proteins of Araneus diadematus. Modified spider silk fibroin derived from natural spider silk fibroin may be modified spider silk fibroin derived from these dragline silk proteins. Modified spider silk fibroin derived from ADF3 has excellent characteristics of being relatively easily synthesized and having excellent strength, elongation, and toughness.

A weft thread protein is produced in the flagelliform gland of spiders. Examples of weft thread proteins include a flagelliform silk protein derived from Nephila clavipes.

Modified spider silk fibroin may be recombinant spider silk fibroin. Examples of recombinant spider silk fibroin include mutants, analogs, or derivatives of natural spider silk fibroin. A suitable example of such modified spider silk fibroin is recombinant spider silk fibroin of large spinneret dragline silk proteins. For example, recombinant spider silk fibroin is produced in several heterologous protein production systems. Methods for producing recombinant spider silk fibroin use transgenic goat, transgenic silkworm, or recombinant plants or mammalian cells.

Recombinant spider silk fibroin can be obtained by, for example, deleting one or a plurality of sequences encoding the (A)ₙ motif from the cloned genetic sequence of naturally occurring fibroin. Alternatively, recombinant spider silk fibroin can be obtained by, for example, designing an amino acid sequence corresponding to an amino acid sequence in which one or a plurality of (A)ₙ motifs have been deleted from the amino acid sequence of naturally-occurring fibroin, and chemically synthesizing a nucleic acid encoding the designed amino acid sequence. In any case, modification on the amino acid sequence corresponding to substitution, deletion, insertion, and/or addition of one or a plurality of amino acid residues may be further performed in addition to the modification corresponding to deletion of the (A)ₙ motifs from the amino acid sequence of naturally-occurring fibroin. Substitution, deletion, insertion, and/or addition of amino acid residues can be carried out by methods well known to those skilled in the art, such as site-directed mutagenesis. Specifically, they can be carried out according to a method described in documents such as Nucleic Acid Res. 10, 6487 (1982), and Methods in Enzymology, 100, 448 (1983).

Examples of recombinant spider silk fibroin of large spinneret dragline silk proteins and modified spider silk fibroin derived from silkworm silk include a protein including a domain sequence represented by Formula 1: [(A)ₙ motif-REP]ₘ. In the (A)ₙ motif in Formula 1, A represents an alanine residue, and n may be an integer of 2 to 27, 2 to 20, 4 to 27, 4 to 20, 8 to 20, 10 to 20, 4 to 16, 8 to 16, or 10 to 16. It is sufficient that the ratio of the number of alanine residues be 40% or more with respect to the total number of amino acid residues in the (A)ₙ motif, and the ratio may be 60% or more, 70% or more, 80% or more, 83% or more, 85% or more, 86% or more, 90% or more, 95% or more, or 100% (which means that the (A)n motif consists of only alanine residues). REP represents an amino acid sequence consisting of 2 to 200 amino acid residues. m represents an integer of 2 to 300. The total number of glycine (Gly) residues, serine (Ser) residues, and alanine (Ala) residues contained in the amino acid sequence represented by Formula 1 is preferably 40% or more and may be 60% or more or 70% or more, with respect to the total number of amino acid residues. The plurality of (A)ₙ motifs may be the same amino acid sequences or different amino acid sequences. A plurality of REP's may be the same amino acid sequences or different amino acid sequences. Specific examples of modified spider silk fibroin derived from large spinneret dragline silk include a protein including the amino acid sequence set forth in SEQ ID NO: 1 or SEQ ID NO: 2.

Examples of fibroin derived from weft thread proteins include a protein including a domain sequence represented by Formula 2: [REP2]ₒ (in Formula 2, REP2 represents an amino acid sequence consisting of Gly-Pro-Gly-Gly-X, and X represents one amino acid selected from the group consisting of Alanine (Ala), Serine (Ser), Tyrosine (Tyr), and Valine (Val). o represents an integer of 8 to 300). Examples of fibroin derived from weft thread proteins include a protein including 10 or more, preferably 20 or more, more preferably 30 or more units of the amino acid sequence represented by Formula 2: REP2. For recombinant protein production using a microorganism such as Escherichia coli as a host, fibroin derived from weft thread proteins may have a molecular weight of 500 kDa or less, 300 kDa or less, or 200 kDa or less from the viewpoint of productivity. Specific examples include a protein including the amino acid sequence set forth in SEQ ID NO: 3. The amino acid sequence set forth in SEQ ID NO: 3 is an amino acid sequence obtained by combining an amino acid sequence (referred to as a PR1 sequence) from the 1220th residue to the 1659th residue from the N-terminus, which corresponds to a repeat moiety and motif of a partial sequence (NCBI Accession No: AAF36090, GI: 7106224) of the flagelliform silk protein of Nephila clavipes obtained from the NCBI database, with a C-terminal amino acid sequence from the 816th residue to the 907th residue from the C-terminus of a partial sequence (NCBI Accession No: AAC38847, GI: 2833649) of the flagelliform silk protein of Nephila clavipes obtained from the NCBI database, and then adding the amino acid sequence (tag sequence and hinge sequence) set forth in SEQ ID NO: 4 to the N-terminus of the combined sequence.

Fibroin can be produced by, for example, expressing a nucleic acid in a host transformed with an expression vector having a nucleic acid sequence encoding a desired protein and one or a plurality of regulatory sequences operably linked to the nucleic acid sequence.

A method for manufacturing a nucleic acid encoding desired fibroin is not particularly limited. The nucleic acid can be manufactured by, for example, a method in which a gene encoding natural fibroin is amplified and cloned by polymerase chain reaction (PCR) or the like; or a method of chemically synthesizing a nucleic acid. A method for chemically synthesizing a nucleic acid is not particularly limited and, for example, genes can be chemically synthesized by a method of linking, by PCR or the like, oligonucleotides that are automatically synthesized by AKTA oligopilot plus 10/100 (GE Healthcare Japan Ltd.) or the like, based on amino acid sequence information of structural proteins obtained from the NCBI web database and the like. In this case, in order to facilitate purification and/or confirmation of proteins, a nucleic acid encoding a protein consisting of an amino acid sequence obtained by adding an amino acid sequence consisting of a start codon and a His10 tag to the N-terminus of the above amino acid sequence may be synthesized.

### EXAMPLES

Hereinafter, the present invention will be described more specifically with reference to examples. However, the present invention is not limited to the following examples.

1. Manufacture of Modified Spider Silk Fibroin

(1) Production of Plasmid Expression Strain

Modified spider silk fibroin (hereinafter, also referred to as “PRT799”) having the amino acid sequence set forth in SEQ ID NO: 5 was designed on the basis of the base sequence and amino acid sequence of fibroin (GenBank Accession No: P46804.1, GI: 1174415) derived from Nephila clavipes. The amino acid sequence set forth in SEQ ID NO: 5 has an amino acid sequence obtained by subjecting the amino acid sequence of fibroin derived from Nephila clavipes to substitution, insertion, and deletion of amino acid residues for the purpose of improving productivity and the amid acid sequence (tag sequence and hinge sequence) set forth in SEQ ID NO: 6 added to the N-terminus of the above amino acid sequence. Modified spider silk fibroin (hereinafter, also referred to as “PRT966”) having the amino acid sequence set forth in SEQ ID NO: 7 was also designed.

A nucleic acid encoding PRT799 or PRT966 was synthesized. In the nucleic acid, an NdeI site was added to the 5′ end and an EcoRI site was added downstream of the stop codon. The nucleic acid was cloned into a cloning vector (pUC118). Thereafter, the same nucleic acid was cleaved by a restriction enzyme treatment with NdeI and EcoRI, and then recombined into a protein expression vector pET-22b(+) to obtain an expression vector.

(2) Expression of Protein

Escherichia coli BLR (DE3) was transformed with the obtained expression vector pET-22b(+). The transformed Escherichia coli was cultured in 2 mL of an LB medium containing ampicillin for 15 hours. The culture solution was added to 100 mL of a seed culture medium (Table 1) containing ampicillin so that the OD600 was 0.005. The temperature of the culture solution was maintained at 30° C., and flask culture was carried out (for about 15 hours) until the OD600 reached 5, thereby obtaining a seed culture solution.

TABLE 1
Seed Culture Medium

| Reagent | Concentration (g/L) |
|---|---|
| Glucose | 5.0 |
| KH₂PO₄ | 4.0 |
| K₂HPO₄ | 9.3 |
| Yeast Extract | 6.0 |
| Ampicillin | 0.1 |

The seed culture solution was added to a jar fermenter containing 500 mL of a production medium (Table 2) so that the OD600 was 0.05. The transformed Escherichia coli was cultured while the temperature of the culture solution was maintained at 37° C. The culture solution was maintained at pH 6.9, and the concentration of oxygen dissolved in the culture solution was maintained at 20% of the saturated concentration of dissolved oxygen.

TABLE 2
Production Medium

| Reagent | Concentration (g/L) |
|---|---|
| Glucose | 12.0 |
| KH₂PO₄ | 9.0 |
| MgSO₄•7H₂O | 2.4 |
| Yeast Extract | 15 |
| FeSO₄•7H₂O | 0.04 |
| MnSO₄•5H₂O | 0.04 |
| CaCl₂•2H₂O | 0.04 |
| GD-113 (Defoamer) | 0.1 (mL/L) |

Immediately after glucose in the production medium was completely consumed, a feed solution (455 g/1 L of glucose and 120 g/1 L of Yeast Extract) was added at a rate of 1 mL/min. Thereafter, the culture was continued at pH 6.9 for 20 hours while the temperature of the culture solution was maintained at 37° C. The concentration of oxygen dissolved in the culture solution was maintained at 20% of the saturated concentration of dissolved oxygen.

Thereafter, 1 M isopropyl-β-thiogalactopyranoside (IPTG) was added to the culture solution so that the final concentration became 1 mM to induce the expression of the target protein. Twenty hours after addition of IPTG, the culture solution was centrifuged to recover the bacterial cells. SDS-PAGE was carried out using the bacterial cells prepared from the culture solution before the addition of IPTG and after the addition of IPTG. The expression of the target modified spider silk fibroin “PRT799” or “PRT966” was confirmed by a band having a size of the target spider silk fibroin appeared according to the addition of IPTG.

(3) Purification of Protein

The bacterial cells recovered 2 hours after the addition of IPTG were washed with 20 mM Tris-HCl buffer (pH 7.4). The bacterial cells after washing were suspended in 20 mM Tris-HCl buffer solution (pH 7.4) containing about 1 mM PMSF, and the cells were disrupted with a high-pressure homogenizer (available from GEA Niro Soavi SpA). The disrupted cells were centrifuged to obtain a precipitate. The obtained precipitate was washed with 20 mM Tris-HCl buffer solution (pH 7.4) until reaching a high purity. The precipitate after washing was suspended in 8 M guanidine buffer solution (8 M guanidine hydrochloride, 10 mM sodium dihydrogen phosphate, 20 mM NaCl, 1 mM Tris-HCl, pH 7.0) so that the concentration became 100 mg/mL. The suspension was stirred with a stirrer at 60° C. for 30 minutes so that the precipitate was dissolved. After dissolution, dialysis was carried out with water using a dialysis tube (cellulose tube 36/32 available from Sanko Junyaku Co., Ltd). The white aggregated protein obtained after dialysis was recovered by centrifugation. The recovered aggregated protein was dried with a freeze dryer to obtain a protein powder containing modified spider silk fibroin “PRT799” or “PRT966”.

2. Fibroin Solution and Protein Fiber

2-1. PRT799

### Example 1-1

Dispersion Step

A mixing device (flow jet mixer MW-J-300-S available from Funken Powtechs, Inc) having a disc-shaped rotary body for mixing was provided. With the rotary body of the mixing device rotating at a rotational speed of 900 rpm, a dissolving liquid at 20° C. containing DMSO and 4% by mass of lithium chloride was continuously supplied onto the rotation surface of the rotary body, whereby a thin film of the dissolving liquid was flowed on the rotation surface. The protein powder containing PRT799 was supplied through a powder inlet above the rotation surface and introduced to the dissolving liquid on the rotation surface. The dissolving liquid was supplied at a flow rate of 15 kg/hr by using a gear pump. The protein powder was supplied at a rate of 5 kg/hr by using a feeder. Accordingly, the flowing dissolving liquid and the protein powder were continuously mixed to form a slurry containing these.

Dissolution Step

The obtained slurry was fed to an in-line mixer (static mixer available from Noritake Co., Ltd.: model SMHEDM-25A(24)/SL) maintained at 80° C. by using a progressing cavity pump. The flow rate of the slurry was 0.3 L/min. Twenty four static mixer elements were previously attached to the in-line mixer. The slurry was stirred and heated in the in-line mixer, whereby the protein powder was dissolved in the dissolving liquid. The time it took for the slurry to pass through the in-line mixer was 1 minute, and the temperature of the fibroin solution discharged from the in-line mixer was 70° C.

Filtration, Defoaming, and Cooling

The obtained fibroin solution was filtered through a sintered metal filter with 1 μm mesh, whereby insoluble matter was removed. Next, the fibroin solution was defoamed by maintaining the fibroin solution under a reduced pressure of −0.1 MPa for 60 minutes. Subsequently, the fibroin solution was cooled to 15° C. by feeding the fibroin solution to an in-line mixer (the same model as descried above) maintained at 10° C. The cooled fibroin solution was fed to a storage tank.

Spinning (Protein Fiber)

A filament containing modified spider silk fibroin was obtained by dry-wet-type spinning using the cooled fibroin solution as a spinning raw liquid. The conditions of dry-wet-type spinning are as described below.
Temperature of coagulation liquid (methanol): 5° C. to 10° C.
Total draw ratio: 5 times
Drying temperature: 80° C.

### Example 1-2

A fibroin solution was prepared through the same dispersion step and the same dissolution step as in Example 1-1 except that formic acid was used as a dissolving liquid instead of the dissolving liquid containing DMSO and 4% by mass of lithium chloride. A filament was produced by using the obtained fibroin solution in the same manner as in Example 1-1.

### Example 1-3

A fibroin solution was prepared in the same manner as in Example 1-1 except that the number of static mixer elements used in the dissolution step was changed to 10. A filament was produced by using the obtained fibroin solution in the same manner as in Example 1-1.

### Example 1-4

A slurry obtained in the same dispersion step as in Example 1-1 was supplied to a 100-L tank with a paddle-type stirring blade. Hot water at 80° C. was flowed through a tank jacket. The protein powder was dissolved in the dissolving liquid by heating the slurry for 60 minutes under stirring with the stirring blade to obtain a fibroin solution. A filament was produced by using the obtained fibroin solution in the same manner as in Example 1-1.

### Comparative Example 1-1

To a 100-L tank with a paddle-type stirring blade, 16 kg of protein powder and 64 kg of a dissolving liquid containing DMSO and 4% by mass of lithium chloride were added. These were stirred in the tank for 60 minutes to obtain a slurry. A fibroin solution was obtained by using the obtained slurry in the same dissolution step as in Example 1-1. A filament was produced by using the obtained fibroin solution in the same manner as in Example 1-1.

### Comparative Example 1-2

A slurry was formed in the tank in the same manner as in Comparative Example 1. Subsequently, hot water at 80° C. was flowed through the tank jacket, and the protein powder was dissolved in the dissolving liquid by heating the slurry for 60 minutes under stirring to obtain a fibroin solution. A filament was produced by using the obtained fibroin solution in the same manner as in Example 1-1.

2-2. PRT966

### Example 2-1

A slurry containing a protein powder and a dissolving liquid was formed in the same dispersion step as in Example 1-1, except that a protein powder containing PRT966 was used instead of the protein powder containing PRT799, and formic acid was used as a dissolving liquid. The supply of the dissolving liquid and formic acid was adjusted such that the concentration of protein in the slurry was 31% by mass based on the mass of the slurry.

The obtained slurry was fed to an in-line mixer (static mixer available from Noritake Co., Ltd.: model SMHEDM-25A(24)/SL) maintained at 80° C. by using a progressing cavity pump. The flow rate of the slurry was 0.3 L/min. Twenty four static mixer elements were previously attached to the in-line mixer. The slurry was stirred and heated in the in-line mixer, whereby the protein powder was dissolved in the dissolving liquid. The time it took for the slurry to pass through the in-line mixer was 1 minute, and the temperature of the fibroin solution discharged from the in-line mixer was 70° C.

A filament was produced by using the obtained fibroin solution in the same manner as in Example 1-1.

### Example 2-2

A slurry obtained in the same dispersion step as in Example 2-1 was supplied to a 100-L tank with a paddle-type stirring blade. Hot water at 40° C. was flowed through a tank jacket. The protein powder was dissolved in the dissolving liquid by heating the slurry for 60 minutes under stirring with the stirring blade to obtain a fibroin solution. A filament was produced by using the obtained fibroin solution in the same manner as in Example 1-1.

### Comparative Example 2-1

To a 100-L tank with a paddle-type stirring blade, 31 kg of protein powder and 69 kg of formic acid were added. These were stirred in the tank for 60 minutes to obtain a slurry. A fibroin solution was obtained by using the obtained slurry in the same dissolution step using the in-line mixer as in Example 2-1. A filament was produced by using the obtained fibroin solution in the same manner as in Example 1-1.

### Comparative Example 2-2

A slurry was formed in the tank in the same manner as in Comparative Example 2-1. Subsequently, hot water at 40° C. was flowed through the tank jacket, and the protein powder was dissolved in the dissolving liquid by heating the slurry for 60 minutes under stirring to obtain a fibroin solution. A filament was produced by using the obtained fibroin solution in the same manner as in Example 1-1.

3. Evaluation

(1) Rate of Lump Generation

A slurry (1000 g) was placed in a container having a volume of 1 L and including a sintered metal filter with 2 mm mesh in a lower part. The slurry was passed through the filter by pressuring the container with 0.05 MPa nitrogen. Lumps in the slurry did not pass through the filter. The mass W₁ (g) of the slurry that had passed through the filter was measured. The rate of lump generation was calculated from Formula: the rate of lump generation={(1000−W)₁/1000}×100.

(2) Dissolution State (Filtration Rate)

The dissolution state of the protein powder in the fibroin solution before the fibroin solution passing through a sintered metal filter was evaluated on the basis of the filtration rate. The fibroin solution (1000 g) was filtered through a sintered metal filter with 4.7 cm² filter area and 1 μm mesh by feeding the fibroin solution by using a metering pump while maintaining the fibroin solution at 40° C. The mass W₂ (g) of the fibroin solution that had passed through the filter was measured. The filtration rate was calculated from Formula: the filtration rate=(W₂/1000)×100. A large filtration rate means a small amount of undissolved fibroin.

(3) Viscosity of Fibroin Solution

The viscosity of the fibroin solution after cooling was measured by using a viscometer (model EMS-01S available from Kyoto Electronics Manufacturing Co., Ltd). The measurement conditions are as described below.
Temperature: 40° C.
Measurement time: 2 minutes
Probe rotation speed: 2000 rpm

(4) Strength and Elongation of Fiber

The opposite ends of the filament were fixed to a test strip with an adhesive, and tensile testing was carried out by using a tensile tester (available from Instron Co., Ltd., model: 3342) under the conditions of a gripping jig distance of 20 mm and a tensile speed of 10 cm/min. Tensile testing was carried out in an environment at a temperature of 20° C. and a relative humidity of 65%. The strength (stress at break) and the elongation (elongation at break) were determined from the obtained stress-strain curve. The strength and elongation shown in Table 3 are relative values based on Comparative Example 1-2. The strength and elongation shown in Table 4 are relative values based on Comparative Example 2-2.

TABLE 3

| | | | | | | Comparative | Comparative |
|---|---|---|---|---|---|---|---|
| | | Example 1-1 | Example 1-2 | Example 1-3 | Example 1-4 | Example 1-1 | Example 1-2 |
| Modified Fibroin | | PRT799 | PRT799 | PRT799 | PRT799 | PRT799 | PRT799 |
| Solvent | | DMSO | formic acid | DMSO | DMSO | DMSO | DMSO |
| Dispersion Step | | thin film | thin film | thin film | thin film | stirring in | stirring in |
| | | | | | | tank | tank |
| Dissolution | Device | in-line | in-line | in-line | stirring in | in-line | stirring in |
| Step | | mixer | mixer | mixer | tank | mixer | tank |
| | Number of | 24 | 24 | 10 | | 24 | |
| | Elements | | | | | | |
| | Time | 1 min | 1 min | 1 min | 60 min | 1 min | 60 min |
| Rate of Lump Generation | | 0 | 0 | 0 | 0 | 18 | 24 |
| [% by mass] | | | | | | | |
| Dissolution State | | 99 | 99 | 99 | 98 | 76 | 71 |
| (Filtration Rate, | | | | | | | |
| [% by mass]) | | | | | | | |
| Solution Viscosity [mPa · s] | | 25000 | 15000 | 24000 | 21000 | 16000 | 9000 |
| Fiber | Strength | 167% | 213% | 160% | 133% | 120% | 100% |
| | Elongation | 194% | 83% | 194% | 106% | 183% | 100% |

TABLE 4

| | | | | | | Comparative | | Comparative | |
|---|---|---|---|---|---|---|---|---|---|
| | | Example 2-1 | | Example 2-2 | | Example 2-1 | | Example 2-2 | |
| Modified Fibroin | | PRT966 | | PRT966 | | PRT966 | | PRT966 | |
| Solvent | | formic acid | | formic acid | | formic acid | | formic acid | |
| Dispersion Step | | thin film | | thin film | | stirring in | | stirring in | |
| | | | | | | tank | | tank | |
| Dissolution | Device | in-line | | stirring in | | in-line | | stirring in | |
| Step | | mixer | | tank | | mixer | | tank | |
| | Number of | 24 | | | | 24 | | | |
| | Elements | | | | | | | | |
| | Time | 1 | min | 60 | min | 1 | min | 60 | min |

| Rate of Lump | 0 | | 1 | | 20 | | 28 | |
|---|---|---|---|---|---|---|---|---|
| Generation [% by mass] | | | | | | | | |
| Dissolution State | 99 | | 97 | | 73 | | 66 | |
| (Filtration Rate, [% | | | | | | | | |
| by mass]) | | | | | | | | |
| Solution Viscosity | 40900 | | 38900 | | 29200 | | 18800 | |
| [mPa · s] | | | | | | | | |
| Draw Ratio | 7 | times | 6.5 | times | 6.2 | times | 6 | times |

| | | | | | |
|---|---|---|---|---|---|
| Fiber | Strength | 123% | 120% | 118% | 100% |
| | Elongation | 54% | 87% | 110% | 100% |

The evaluation results shown in Table 3 and Table 4 indicate that generation of lumps in forming the slurry is sufficiently suppressed according to the methods of Examples including the dispersion step for continuously forming the slurry while flowing the thin film of the dissolving liquid. In each Example, the fibroin solution with less undissolved matter was easily obtained by dissolving, in the solvent, the protein powder in the slurry. In addition, the protein fiber having good mechanical properties was formed from the fibroin solution of each Example. The mechanical properties of the protein fiber were particularly preferable when the protein fiber was manufactured by using the fibroin solutions of Examples 1-1 to 1-3 in which the dispersion step for forming the slurry while flowing the thin film of the dissolving liquid is combined with the dissolution step in the in-line mixer.

For the fiber of PRT966, the elongation in Examples 2-1 and 2-2 tends to be smaller than that in Comparative Examples 2-1 and 2-2, which mainly results from the draw ratio being relatively larger in Examples 2-1 and 2-2. In Examples 2-1 and 2-2, the drawability increases as the solubility of fibroin increases, which results in a high draw ratio. With a high draw ratio, a fiber having a higher strength can be obtained. A fiber having a higher elongation may be obtained by setting the draw ratio to a lower value in Examples 2-1 and 2-2.

### REFERENCE SIGNS LIST

1 Protein powder
3 Dissolving liquid
3a Thin film
5 Slurry
7 Fibroin solution
10 Rotary body
11 Shaft
12 Controller
20 Mixing device
21 Dissolving liquid inlet
23 Feed pipe
25 Retention tank
25A Bottom surface
25B Inner circumferential surface
30 Powder inlet
40 In-line mixer
41 Pipe
43 Static mixer element
45 Jacket
45a Heating medium inlet
45b Heating medium outlet
S Rotation surface

### SEQUENCE LISTING

1. A method for manufacturing a fibroin solution, the method comprising:
a step of forming a slurry containing a solvent-containing dissolving liquid and a fibroin-containing protein powder dispersed in the dissolving liquid by continuously introducing the protein powder to a thin film of the dissolving liquid while flowing the thin film; and
a step of forming a fibroin solution by dissolving, in the dissolving liquid, the protein powder in the slurry.

2. The method according to claim 1, wherein the thin film of the dissolving liquid is flowed on a surface by continuously supplying the dissolving liquid onto the surface.

3. The method according to claim 1, wherein the step of forming the slurry includes continuously supplying the dissolving liquid onto a rotation surface of a rotary body by using a metering pump so that the thin film of the dissolving liquid flows on the rotation surface, and continuously supplying the protein powder by using a metering feeder so that the protein powder is continuously introduced to the thin film on the rotation surface.

4. The method according to claim 1, wherein the protein powder in the slurry is dissolved in the dissolving liquid by heating the slurry under stirring in an in-line mixer.

5. The method according to claim 1, further comprising a step of cooling the fibroin solution under stirring in the in-line mixer.

6. The method according to claim 1, further comprising a step of defoaming the fibroin solution.

7. The method according to claim 1, further comprising a step of removing insoluble matter by filtering the fibroin solution.

8. The method according to claim 1, wherein the solvent contains at least one selected from the group consisting of dimethyl sulfoxide, formic acid, and hexafluoroisopropanol.

9. The method according to claim 1, wherein the dissolving liquid further contains an inorganic salt.

10. The method according to claim 1, wherein the fibroin is modified fibroin.

11. The method according to claim 10, wherein the modified fibroin is modified spider silk fibroin.

12. A method for manufacturing a protein molded body, the method comprising:
producing a fibroin solution by the method according to claim 1; and
producing a fibroin-containing protein molded body by molding using the fibroin solution as a raw liquid for molding.

13. The method according to claim 12, wherein the protein molded body is a protein fiber.


SEQUENCE LISTING (7 sequences)

SEQ ID NO: 1 - protein, 597 aa, Artificial Sequence
  Met-PRT313
MGPGGQGPYGPGASAAAAAGGNGPGSGQQGPGGSAAAAAGGYGPGGQGPGQQGPGSSAAAAAGPGGYGPGGQGPSASAAAAAGPGSGQQGPGASAAAAAGGYGPGGQGPGQQGPGSSAAAAAGGYGSGPGQQGPYGSAAAAAGPGSGGYGQGPYGPGASAAAAAGPGGYGPGGQGPSASAAAAAGSGQQGPGGYGPYASAAAAAGGYGSGPGQQGPYGPGGSAAAAAGSGQQGPGQQGPYASAAAAAGPGGQGPYGPGSSAAAAAGGYGYGPGGQGPYGPGASAAAAAGGNGPGSGGYGPGQQGPGGSAAAAAGPGGQGPYGPGASAAAAAGGYGPGGQGPGGYGPGSSAAAAAGPGGQGPYGPGSSAAAAAGGYGPGQQGPYGPGGSAAAAAGGYQQGPGGQGPYGPGASAAAAAGPGGQGPYGPGASAAAAAGPGGYGPGGQGPSASAAAAAGGYGSGPGGYGPYGPGGSAAAAAGPGSGQQGQGPYGPGASAAAAAGGYGPGQQGPYGPGGSAAAAAGPGSGGYGPGASAAAAAGGNGPGSGGYGPGQQGPGGSAAAAAGGYQQGPGGQGPYGPGASAAAAAGPGSGQQGPGAS

SEQ ID NO: 2 - protein, 590 aa, Artificial Sequence
  Met-PRT399
MGPGGQGPYGPGASAAAAAGGNGPGSGQQGPGGSGGYGPGGQGPGQQGPGSSAAAAAGPGGYGPGGQGPSASAAAAAGPGSGQQGPGASGGYGPGGQGPGQQGPGSSAAAAAGGYGSGPGQQGPYGSAAAAAGPGSGGYGQGPYGPGASGPGGYGPGGQGPSASAAAAAGSGQQGPGGYGPYASAAAAAGGYGSGPGQQGPYGPGGSGSGQQGPGQQGPYASAAAAAGPGGQGPYGPGSSAAAAAGGYGYGPGGQGPYGPGASGGNGPGSGGYGPGQQGPGGSAAAAAGPGGQGPYGPGASAAAAAGGYGPGGQGPGGYGPGSSGPGGQGPYGPGSSAAAAAGGYGPGQQGPYGPGGSAAAAAGGYQQGPGGQGPYGPGASGPGGQGPYGPGASAAAAAGPGGYGPGGQGPSASAAAAAGGYGSGPGGYGPYGPGGSGPGSGQQGQGPYGPGASAAAAAGGYGPGQQGPYGPGGSAAAAAGPGSGGYGPGASGGNGPGSGGYGPGQQGPGGSAAAAAGGYQQGPGGQGPYGPGASAAAAAGGYGSGPGQQGPYGPGGSGSGQQGPGQQGPYASAAAAAGPGSGQQGPGAS

SEQ ID NO: 3 - protein, 559 aa, Artificial
  Recombinant spider silk protein Flag_92_short2
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 4 - protein, 24 aa, Artificial Sequence
  His tag and start codon
MHHHHHHHHHHSSGSSLEVLFQGP

SEQ ID NO: 5 - protein, 2375 aa, Artificial Sequence
  PRT799
MHHHHHHSSGSSGPGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGPGASGQQGPYGPGASAAAAAGQNGPGSGQQGPGQSGQYGPGQQGPGQQGPGSSAAAAAGPGQYGPGQQGPSASAAAAAGPGSGQQGPGASGQYGPGQQGPGQQGPGSSAAAAAGQYGSGPGQQGPYGSAAAAAGPGSGQYGQGPYGPGASGPGQYGPGQQGPSASAAAAAGSGQQGPGQYGPYASAAAAAGQYGSGPGQQGPYGPGQSGSGQQGPGQQGPYASAAAAAGPGQQGPYGPGSSAAAAAGQYGYGPGQQGPYGPGASGQNGPGSGQYGPGQQGPGQSAAAAAGPGQQGPYGPGASAAAAAGQYGPGQQGPGQYGPGSSGPGQQGPYGPGSSAAAAAGQYGPGQQGPYGPGQSAAAAAGQYQQGPGQQGPYGPGASGPGQQGPYGPGASAAAAAGPGQYGPGQQGPSASAAAAAGQYGSGPGQYGPYGPGQSGPGSGQQGQGPYGPGASAAAAAGQYGPGQQGPYGPGQSAAAAAGPGSGQYGPGASGQNGPGSGQYGPGQQGPGQSAAAAAGQYQQGPGQQGPYGPGASAAAAAGQYGSGPGQQGPYGPGQSGSGQQGPGQQGPYASAAAAAGPGSGQQGSSVDKLAAALEHHHHHH

SEQ ID NO: 6 - protein, 12 aa, Artificial Sequence
  His6Tag plus Hinge
MHHHHHHSSGSS

SEQ ID NO: 7 - protein, 1190 aa, Artificial Sequence
  PRT966
MHHHHHHSSGSSGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGASGPGVFGPYGPGASAAAAAGINGPGSGVFGPGISGIYGPGVFGPGVFGPGSSAAAAAGPGIYGPGVFGPSASAAAAAGPGSGVFGPGASGIYGPGVFGPGVFGPGSSAAAAAGIYGSGPGVFGPYGSAAAAAGPGSGIYGIGPYGPGASGPGIYGPGVFGPSASAAAAAGSGVFGPGIYGPYASAAAAAGIYGSGPGVFGPYGPGISGSGVFGPGVFGPYASAAAAAGPGVFGPYGPGSSAAAAAGIYGYGPGVFGPYGPGASGINGPGSGIYGPGVFGPGISAAAAAGPGVFGPYGPGASAAAAAGIYGPGVFGPGIYGPGSSGPGVFGPYGPGSSAAAAAGIYGPGVFGPYGPGISAAAAAGIYVFGPGVFGPYGPGASGPGVFGPYGPGASAAAAAGPGIYGPGVFGPSASAAAAAGIYGSGPGIYGPYGPGISGPGSGVFGIGPYGPGASAAAAAGIYGPGVFGPYGPGISAAAAAGPGSGIYGPGASGINGPGSGIYGPGVFGPGISAAAAAGIYVFGPGVFGPYGPGASAAAAAGIYGSGPGVFGPYGPGISGSGVFGPGVFGPYASAAAAAGPGSGVFGPGAS